(19) United States
(12) Patent Application Publication
(10) Pub. No.: US 2022/0101212 A1
(43) Pub. Date: Mar. 31, 2022

(54) SYSTEM AND METHODS FOR GREENHOUSE GAS EMISSION MODELING AND CALCULATION TO ESTIMATE THE PRODUCT CARBON FOOTPRINT

(71) Applicant: SAP SE, Walldorf (DE)

(72) Inventors: Astrid Graeber, Walldorf (DE); Jochen Mayerle, Flein (DE)

(21) Appl. No.: 17/039,439

(22) Filed: Sep. 30, 2020

(51) Int. Cl. G06Q 10/04; G01N 33/00

(52) U.S. Cl. CPC G06Q 10/04; G01N 33/0062

(57) ABSTRACT

In one aspect, there is disclosed a computer-implemented method that includes extending an existing first data model of an enterprise resource planning system to include a second data model, wherein the first and second data models provide a greenhouse gas emissions model; providing the extended greenhouse gas emissions model to enable a calculation that allocates to the product or the service an amount of greenhouse gas emissions; and causing display of a user interface including the amount of greenhouse gas emissions allocated to the product or the service. Related systems, methods, and articles of manufacture are also disclosed.

### TECHNICAL FIELD

The subject matter described herein relates generally to data processing and, in particular, to modeling greenhouse gas emissions.

### BACKGROUND

Many organizations may rely on enterprise software applications including, for example, enterprise resource planning (ERP) software, customer relationship management (CRM) software, and/or the like. These enterprise software applications may provide a variety of functionalities including, for example, invoicing, procurement, payroll, time and attendance management, recruiting and onboarding, learning and development, performance and compensation, workforce planning, production, logistics, procurement, and/or the like. Some enterprise software applications may be hosted by a cloud-computing platform such that the functionalities provided by the enterprise software applications may be accessed remotely by multiple end users. For example, an enterprise software application may be available as a cloud-based service including, for example, a software as a service (SaaS) and/or the like.

### SUMMARY

In one aspect, a computer-implemented method includes extending an existing first data model of an enterprise resource planning system to include a second data model, wherein the first and second data models provide a greenhouse gas emissions model; providing the extended greenhouse gas emissions model to enable a calculation that allocates to the product or the service an amount of greenhouse gas emissions; and causing display of a user interface including the amount of greenhouse gas emissions allocated to the product or the service.

In some variations one or more of the following can optionally be included. The first data model includes transactional data associated with the product or the service. The second data model includes energy data and flows associated with the product or the service. The second data model includes emissions that occur during a least a portion of a lifecycle of the product or the service, the portion including emissions related to distribution, storage, usage, and/or disposal. The first and second data models are configured to form a directed graph to enable the calculation that allocates to the product or the service the amount of greenhouse gas emissions. The user interface is configured as a Sankey diagram.

Articles are also described that comprise a tangibly embodied machine-readable medium operable to cause one or more machines (e.g., computers, etc.) to result in operations described herein. Similarly, computer systems are also described that may include a processor and a memory coupled to the processor. The memory may include one or more programs that cause the processor to perform one or more of the operations described herein.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a high-level view of an ERP data model including the GHG data model;

FIG. 2A depicts a bakery example of an ERP data model including the GHG data model;

FIGS. 2B-2D depicts an example of process for calculating the GHG footprint based on the GHG data flow model;

FIG. 3 depicts an example of the GHG data model;

FIG. 4 depicts an example of allocating GHG emissions based on a gas consumption;

FIG. 5 depicts an example of allocating GHG emissions based on an electricity consumption;

FIGS. 6-11 depict examples of user interfaces at which one or more aspects of the GHG data model are defined;

FIGS. 12 and 13 depict user interfaces showing examples of results of a GHG emissions allocation;

FIG. 14 depicts an example of a process for allocating GHG emissions based on the GHG data model layer and the ERP data model; and

FIG. 15 shows a diagram of a system consistent with implementations of the current subject matter.

When practical, similar reference numbers denote similar structures, features, or elements.

### DETAILED DESCRIPTION

Fighting climate change may begin with understanding of the anthropogenic greenhouse gas (GHG) emissions footprint imposed by a business or its products. Greenhouse gas emissions do not just happen, but instead they are a result of many distributed processes on all levels of an enterprise system. To enable an enterprise system to provide meaningful information (which can enable better decisions and processes that minimize an enterprise's greenhouse gas emission footprint), the enterprise needs automated tools to analyze the impact of the enterprise system including production, transportation, logistics, and the embedded contribution of procured materials, components, and energy to the overall greenhouse gas balance of services and finished goods.

In some embodiments, there is provided a GHG data model which is used to extend the data models of an enterprise system, such as an enterprise resource planning (ERP) system, so that the enterprise system can allocate GHG emissions to products or services.

In order to take informed decisions on a product, a proportional quantity of GHG emissions for a product may be estimated, in accordance with some embodiments. Like the costing analytics provided by an ERP system, one or more GHG data models may extend the models of an ERP system to enable allocation of GRG emissions to a given product or product line. Disclosed herein is a system and method for using GHG data models with an ERP system to allocate GHG emission to one or more products.

A problem with respect to GHG data modeling is how to assign GHG emissions on an individual product level captured from different sources (e.g., energy providers, suppliers, transportation, subcontracting, others) while keeping the assignment to a product as true to source as possible, with minimal (if any) manual, user-effort, and do this in a repeatable or auditable way. Moreover, the GHG data modeling may also take into account protocols and standards, such as Greenhouse Gas Protocol: Product Life Cycle Accounting and Reporting Standard, GHG protocol guidance. (See, e.g., https://ghgprotocol.org/sites/default/files/standards/Product-Life-Cycle-Accounting-Reporting-Standard_041613.pdf, date visited Aug. 6, 2020).

In some embodiments, the GHG data models (which are used in conjunction with the ERP system to allocate GHG costs to a given product (or product line)) may include a unified computation of GHG emission categories and an enterprise, system wide consistent GHG data model.

In some embodiments, the GHG data model includes nodes and connectors representative of flows. The nodes may indicate a value, such as a quantity of energy consumed at a given time t1, for example. The nodes and flows may be configured as a directed graph. This directed graph may be made up of a set of nodes connected by connectors (e.g., edges) in which the edges have a direction associated with the flow of that edge. The ERP system may also model a production process using an ERP data model having nodes and connectors. The ERP data model including the GHG data model allows allocating resources including the GHG emissions at a product level using the existing ERP system infrastructure.

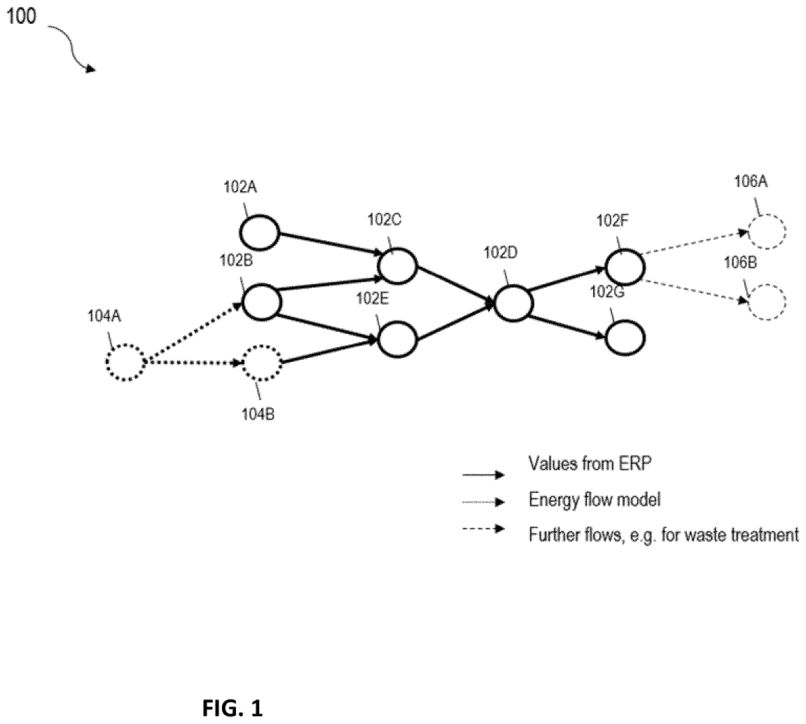

FIG. 1 depicts a high-level view of an ERP data model including a GHG data model 100. In the example of FIG. 1, the ERP system's data flows and nodes represent the material, substance, and process flow(s) in a company. The data flows and nodes of the ERP system are modeled at 102A-G (as indicated by the solid lines). The GHG data model includes nodes and data flows 104A-B (as indicated by the dotted lines). In the example of FIG. 1, the disposal aspects of the ERP system may also modeled as shown by the further data flows and nodes at 106A-B (as indicated by the dashed lines). Alternatively, or additionally, the ERP system may also model emissions that occur during the life cycle of a material or a product after the sale, such as emissions related to distribution, storage, product use, disposal, and/or end-of-life. As a result, ERP data model including the GHG data model 100 represents a single, unified model of the physical value chain and the energy flows that allows allocating resources including the GHG emissions at a product level using the existing ERP system infrastructure from cradle to grave, for example.

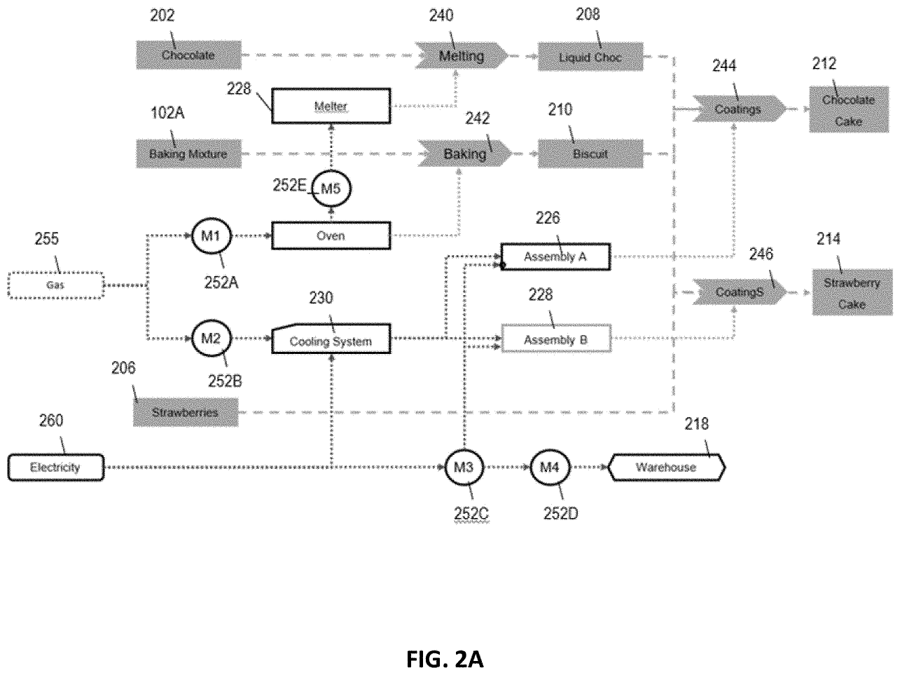

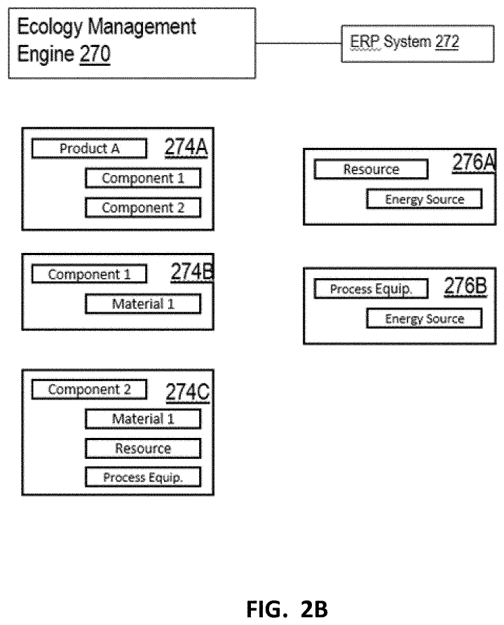

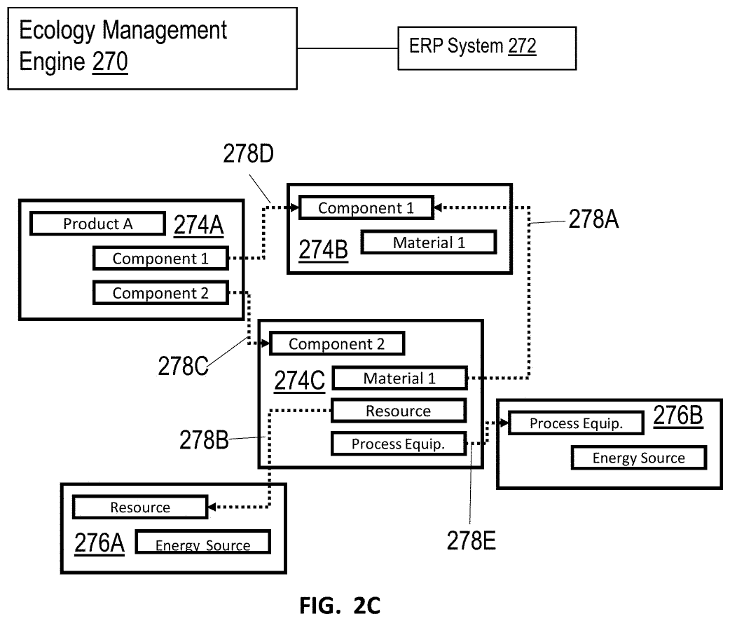

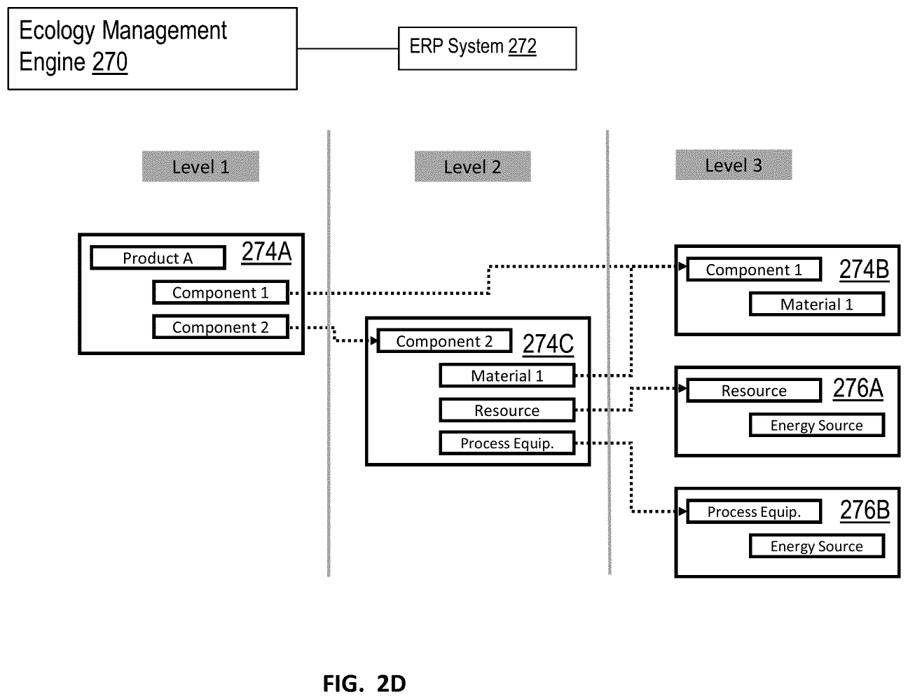

FIG. 2A depicts an example of an ERP data model including a GHG data model 200 generated for a bakery that produces chocolate cake 212 and strawberry cake 214. The example of FIG. 2A is provided merely as an illustrative example to show how the GHG data model can be used to extend the ERP data model to allocate the GHG emissions at a product level using the existing ERP system infrastructure.

The ERP data model nodes may provide ERP data regarding the amounts of materials, such as the amounts of chocolate 202, baking mixtures 204, strawberries 206, liquid chocolate 208, biscuit 210, chocolate cake produced 212, and strawberry cake produced 214. The ERP data flow nodes also provide data regarding the resources in the production process, such as a melter 220, an oven 222, an assembler A 226, an assembler B 228, and a cooling system 230 as well as their time of use. The ERP nodes may also provide a model of the overall production process by depicting certain steps in the process such as melting 240, backing 242, coatings 244 and 246, and warehousing 248. Over time, the nodes of the ERP data model may periodically provide transactional data regarding goods movement during production, such as an opening balance of a good, goods issued, goods received in transit, goods returned, etc. In this way, the ERP data model can determine resources allocated (and thus consumed) to produce a given product or products, such as a chocolate cake at 212 or a strawberry cake 214. However, the ERP data model does not provide any indication of the GHG emissions that should be allocated to the product. To that end, the GHG data flow model is provided as a way to extend the models of the ERP system to take into account GHG emission on a product or service level.

The GHG data flow model may, as noted, extend the ERP data model nodes 202-248. In the example of FIG. 2, the GHG data flow model may include meters 252A-E that measure or indicate energy usage over time. The GHG data flow model may also include nodes for the energy resources being used, such as gas 255 and electricity 260.

During cake production for example, the meter 252A may periodically provide values indicative of the gas being consumed by the oven 222. Likewise, the meter 252B may periodically provide values indicative of the gas being consumed by the cooling system 230, and meter 252E provides an indication of the gas consumed by melter 228. During the production of the cakes 212/214, the meter 252C may periodically provide values indicative of the electricity being used by assemblers A and B 226/228, while meter 252D may periodically provide an indication of the electricity used for warehousing 218 of the cakes or the materials used in their production. If a meter is not available for a given device, such as a cooling system 230, the GHG data model may include consumption rates, so that the consumption can be determined based on ERP data indicating usage time. For example, ERP data may indicate that a cooling system was “on” for 3 hours during the production of strawberry cake. In this example, the GHG data model having the gas and electrical consumption rate for the cooler 230 can estimate the gas and electricity usage for the cooler during the strawberry cake production.

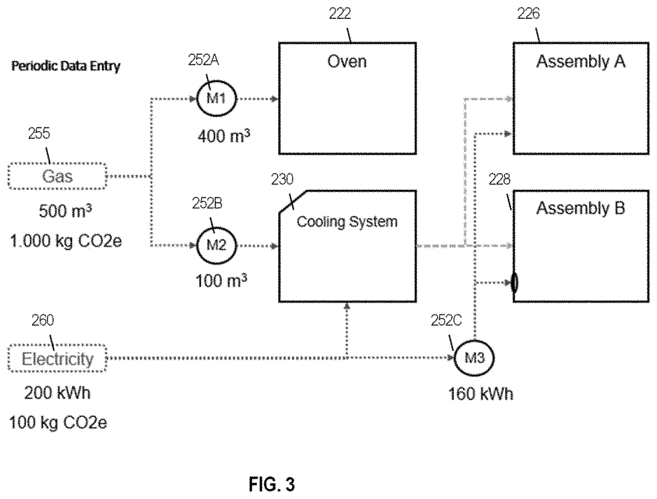

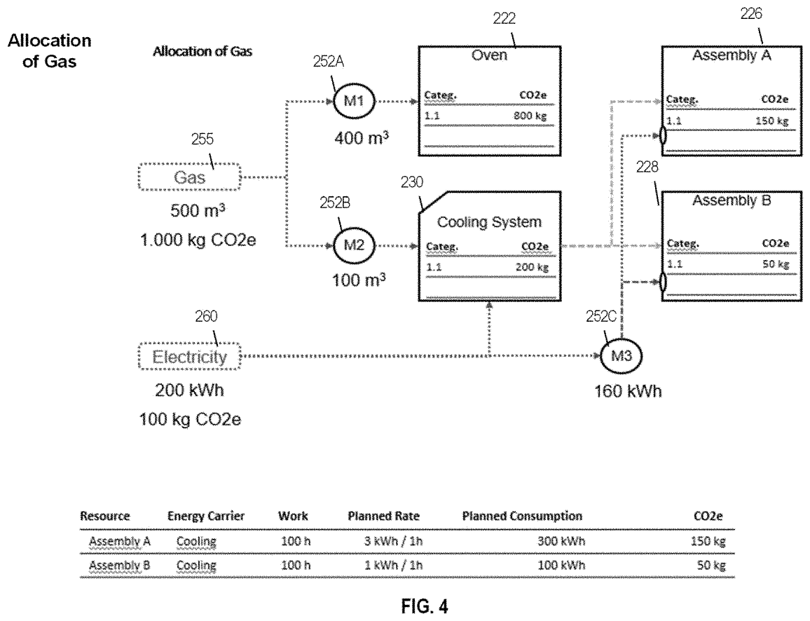

FIG. 3 depicts an example of the GHG data model's nodes 252-260 which provide data periodically regarding energy consumption. For example, the bakery may know its total gas consumption as shown by the gas node 255, which at a given instant in time, t1, there is a consumption of 500 cubic meters of gas with 1,000 kilograms of corresponding CO2 emissions. Because the meter 252A monitor energy consumption of the oven 222 and meter 252B monitors gas consumption of the cooling system 230, the GHG data model can allocate an energy consumption usage of 400 cubic meters of gas consumed by the oven 222 and 100 cubic meters of gas consumed by the cooling system 230. FIG. 4 depicts the allocation, based on gas, of C02 emissions. In this example, the 1000 kilograms of C02 emissions are allocated based on the corresponding consumption of the oven and cooling system, so 800 kilograms of carbon dioxide (C02) emissions are allocated to the oven 222 and 200 kilograms of C02 emissions are allocated to the cooling system. The 150 kg CO2 emissions and the 50 kg CO2 emissions are from the combustion of gas in the cooling system. The 200 kg of CO2 emissions from the cooling system 230 are allocated to assembly A 226 and assembly B 228 by the planned consumption from the ERP system. The allocation may be in accordance with GHG scope category 1.1 stationary combustion.

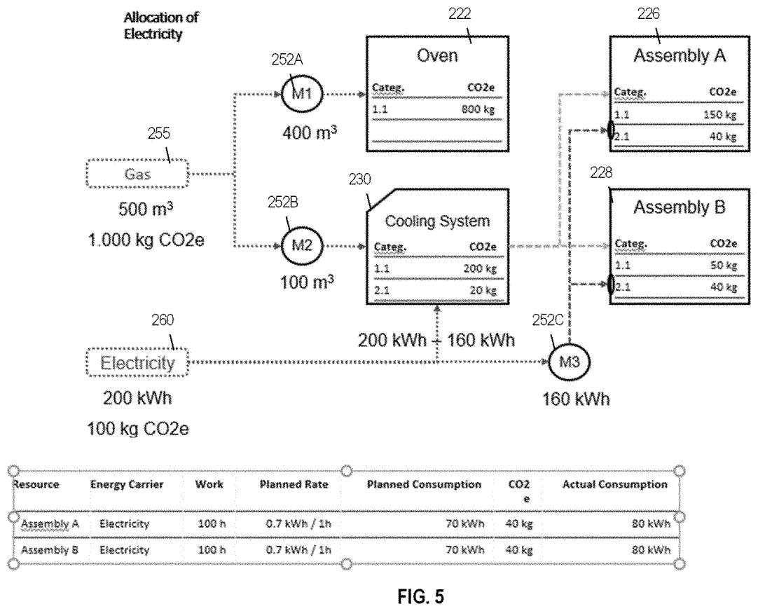

FIG. 5 depicts an example of the CO2 emissions allocation of electricity to each of the assemblers 226 and 228 as well as the cooling system 230. This allocation may be performed at level GHG scope category 2.1, for example. In the example of FIG. 5, the meter 252C monitors electricity consumption of the assemblers 226/228, so the GHG data model can be used to allocate 160 kilowatt-hour (kWh) to each of the assemblers. In the case of electricity, the assemblers 226/228 are allocated only 160 kWh of the 200 kWh overall consumption, the remaining electricity of 40 kWh is attributed to cooling system 230. In this example, the 100 kilograms of C02 emissions are allocated based on the corresponding consumption of the assemblers and the cooling system, so 40 kilograms of C02 emission is allocated to each of the assemblers 226/228 and 20 kilograms of C02 is allocated to the cooling system 230. In this way, the GHG data model may be used to allocate C02 emissions to the production process.

Referring again to FIG. 2A, the allocation of C02 emissions to the oven 222, cooling system 230, and assemblers 226/228 may be used, based on the ERP data model extended to include a GHG data model, to allocate the C02 emission to each of the product lines and corresponding products. In an example where the bakery runs 50% of the time to make 1000 chocolate cakes 212 and the other 50% of the time to make 1000 strawberry cakes 214 (as determined from the ERP data model), the total C02 emissions of 1,100 kilograms of CO2 can be allocated, based on the GHG data model, as 50% (550 kilograms of C02 emissions) to the strawberry cakes and 50% (550 kilograms of C02 emissions) to the chocolate cakes. On a per cake basis, a single chocolate cake is allocated 0.55 kilograms of C02 emissions.

The prior cake allocation example is merely a simplified example for illustrating the CO2 emissions allocation to a product line or a single product. In practice, the GHG data model and the ERP system model may be more complex and the allocation among product lines more complex as well. Indeed, the complexity and dynamic nature of the data and calculation make the task of GHG emission allocation to a product/service impossible with the use of an automated machine, such as an ERP system.

In a first stage, a top-down distribution of the product quantity from the top node, such as the finished product, is allocated along the edge nodes down to the subjacent nodes. At the second stage, a roll-up may be performed of the GHG quantities as a scalar from the lowest level nodes, such as leaf nodes, along all overlying up to the top node, such as the finished product. In a third stage, there may be maintained multiple, parallel GHG node breakdowns as vectors on top of GHG quantity scalar and as explanation component of the GHG origin by GHG quantity break-down by GHG emission categories, GHG quantity break-down by country of emission origin, and GHG quantity break-down by country of emission origin and by emission categories (cross product). And, in the fourth stage, iteratively solving for the network nodes in the directed graph.

The GHG emissions allocation calculations may be performed in a so-called bottom up approach. For example, the CO2 emission values may be rolled up from raw materials and energy sources at a lower level to a finished product level. FIGS. 2B-2D depict an example process for performing the GHG emissions allocation calculations.

FIG. 2B depicts an example of an Ecology Management Engine (EME) 270 retrieving data from an ERP system 272. For example, the EME reads aggregated transaction data from ERP for a given time period. This transaction data may include goods receipt from supplier per material, internal service confirmations from production, and the like. The ECE may create/update instances of product footprint estimates data objects based on the transaction data. Examples of these product emissions footprint estimates data objects are depicts at 274A-C. Next, the EME may create/update the product footprint estimates data objects based on the energy flow model and other data recorded in the footprint inventory. In the example of FIG. 2B, the EME may create/update the product footprint estimates data objects 276A-B based on the energy flow model and other data recorded in the footprint inventory.

FIG. 2C depicts the product footprint estimates data objects being linked. For example, the EME may link 278A-E the instances of the product footprint estimates data objects 272A-C and 276A-B. These links may be implemented using a key or a unique identifier.

FIG. 2D depicts that the linked data objects are then analyzed. Specifically, the actions associated with the data objects may determine levels as shown in FIG. 2D. The EME may then perform a bottom-up calculation. For example, the CO2 emission values may be rolled up from raw materials and energy sources at level 3 to the finished product at level 1.

The EME 270 depicted at FIGS. 2B-D may be provided as a service separate to the ERP system 272 as shown in FIGS. 2B-D or may be included as a component or sub-system of the ERP system.

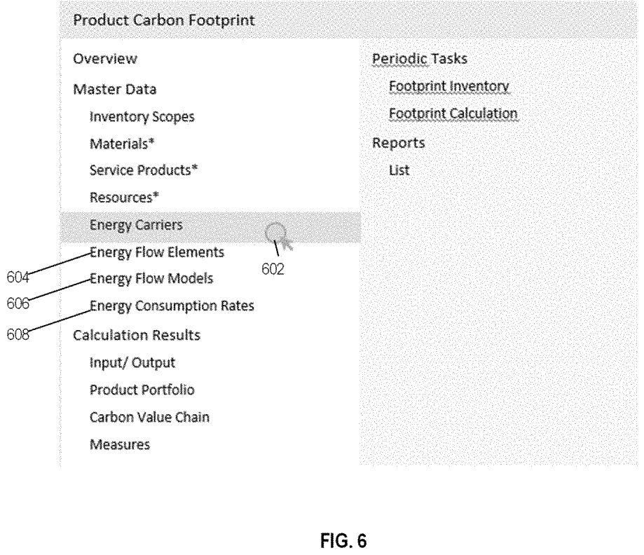

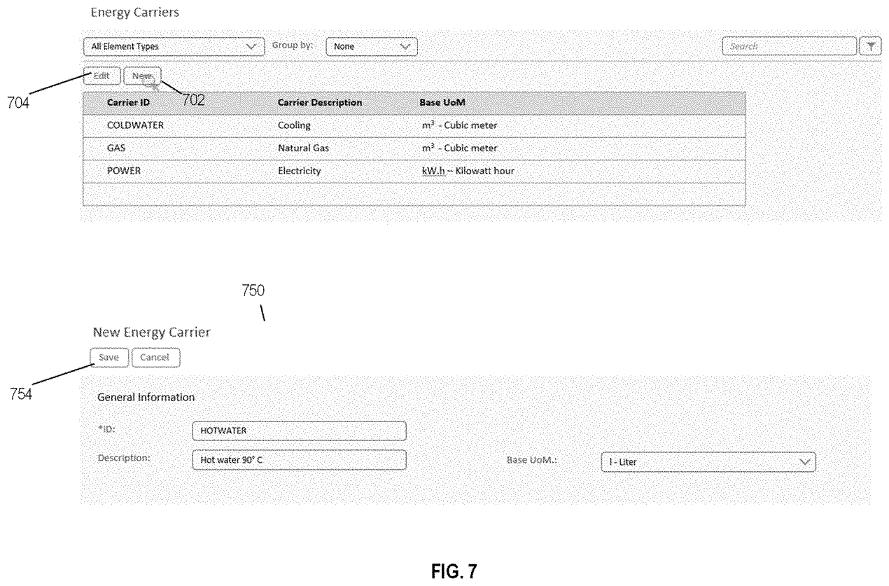

In some implementations, there may be provided one or more user interfaces to enable a user to edit and/or create nodes and the flows for the GHG data model. FIG. 6 depicts an example of a user interface where a user may choose to add energy carriers 602, flow elements 604, flow models 606, or energy consumption rates 608 to the GHG data flow model. Referring to FIG. 6, a selection 602 at a client device may be received, and this selection 602 may be indicative of an energy carrier being selected for use in the model. In response to the selection 602, a user interface 700 at FIG. 7 may be caused to be presented at the client device. The user interface 700 enables editing 704 of an existing energy carrier or defining a new 702 energy carrier, such as a carrier providing cold water, gas, power, steam, etc., each of which may have a unit of measure (UoM).

In response to a new 702 carrier being selected, a user interface 750 may be caused to be presented at the client device. In the example of user interface 750, a hot water energy carrier is newly added, so data may be provided defining that carrier (e.g., an ID “HOTWATER”, a Description “Hot water 90° C.” and a base UoM of 1 liter). After the carrier is defined, the definition may be saved 754 to enable use with the GHG data model. For example, the energy carrier defined at 700 and 750 may be the source of the electricity 260 or gas 255 depicted at the model of FIG. 2, for example.

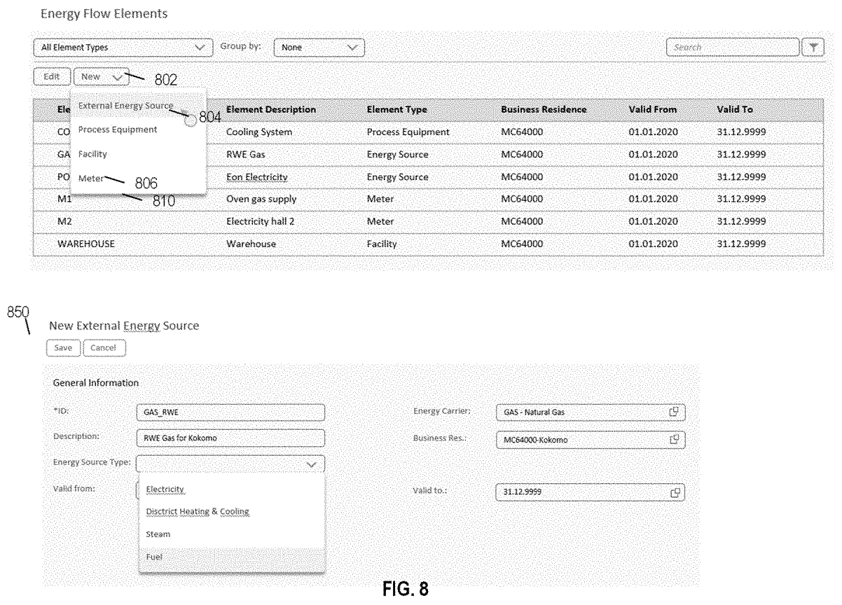

Referring again to FIG. 6, when the energy flow elements is selected at 604, user interface 800 at FIG. 8 is caused to be presented at the client device. The user interface 800 enables edits or creation 802 of a GHG data model element which can be used in the GHG data model of FIG. 2, for example. If a new element is selected 802, a drop down user interface element 810 is presented to enable choosing among elements (e.g., nodes) of the GHG data model, such as an external energy source, a process equipment, a facility, and a meter. The external energy source represents the external energy carrier for different energy types like electricity, fuel, steam or heating/cooling, the process equipment represents, a facility represents auxiliary production equipment, required for the production materials and process flow, but not direct related to the production of finished products, and a meter represents consumption meters that measure energy consumption.

If the external energy source 804 is selected, the user interface 850 is triggered for presentation. At user interface 850, the selected element may be defined by, for example, providing an ID (e.g., “HAS_RWE”), a description of the element (e.g., “RWE Gas for Kokomo”), Energy Source Type (e.g., “Electricity,” Gas, etc.), dates the element are valid to and from, and a type of Energy Carrier (e.g., “GAS_Natural Gas”).

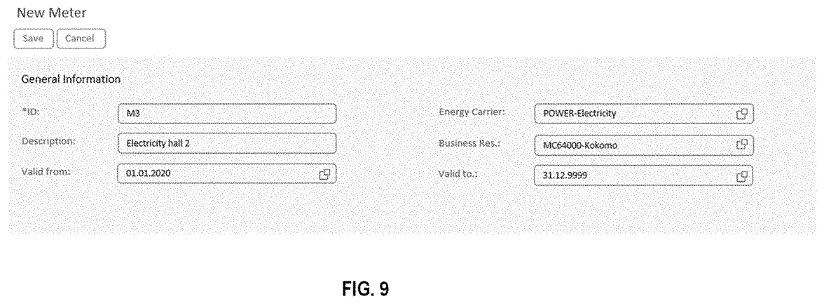

Referring again to user interface 800, if a new meter 806 is selected, a user interface 900 is triggered for presentation as shown at FIG. 9. The user interface 900 enables defining a new meter element. The definition may include providing an ID (e.g., “M3”), a description of the element (e.g., “Electricity hall 2”), a type of Energy Carrier (e.g., “POWER-Electricity”), dates the meter element are valid to and from, a Business Res. information indicative of the physical location of the meter.

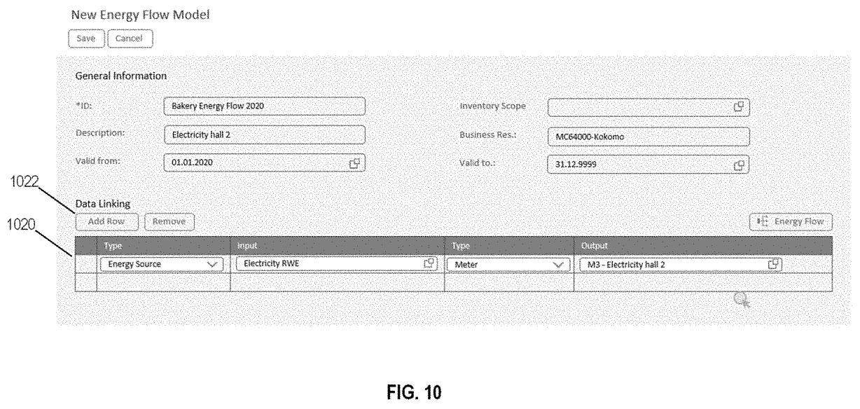

Referring again to FIG. 6, when the energy flow models is selected at 606, the user interface 1000 at FIG. 10 is caused to be presented at the client device. The user interface 1000 allows defining a GHG data model by providing an ID for the model (e.g., “Bakery Energy Flow 2020”), a description of the model, a date range for the validity of the model, an inventory scope indicating footprint inventory periodicity (i.e. valid from, valid to), the companies incorporated into that scope, estimation level (individual product level or product category level) the GHG categories that shall be taken into consideration, and/or a Business Res indicating the physical location.

In the example of FIG. 10, the nodes and flows of the GHG data model may be defined at 1020. For example, a node may be added 1022 at add row. In the example if 1020, the Energy Source node (see, e.g., FIG. 2 or 5 at 260) is being added. By defining the input such as the energy carrier and the output such as the meter (e.g., FIG. 2 or 5 meter 252C), each node of the GHG data model may be defined. The defined GHG data model may form a directed graph, for example. When the GHG data model is defined, it can be run from time to time to generate data indicating the CO2 emissions allocation.

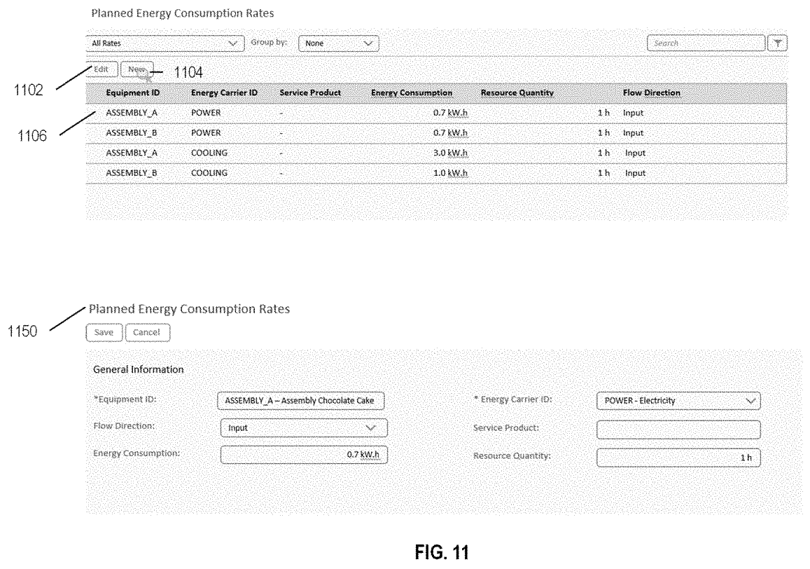

Referring again to FIG. 6, when the energy consumption rates is selected at 608, the user interface 1100 at FIG. 11 is caused to be presented at the client device. The user interface 1100 enables edits 1102 or a new creation 1104 of a consumption rates for the GHG data model node, which can be used in the GHG data model of FIG. 2, for example. If an Assembly A (see, e.g., 226 at FIG. 2) is selected 1106, the user interface 1150 is caused to be presented at the client device. The user interface 1150 enables definitions for equipment ID to which the consumption rate applies (e.g., Assembly A-Assembly Chocolate Cake), flow direction (e.g., input or output) indicating whether this device consumes or produces (energy) flows, energy consumption for the device, such as 0.7 kWh, energy carrier ID (e.g., POWER-COMPANY ABC Electricity), service product indicating the service provided by a specific resource (e.g. Service A: baking at 100 degrees Celsius, baking at 200 degrees Celsius, etc.), and resource quantity indicating the amount of time in which the equipment consumes the given amount of energy.

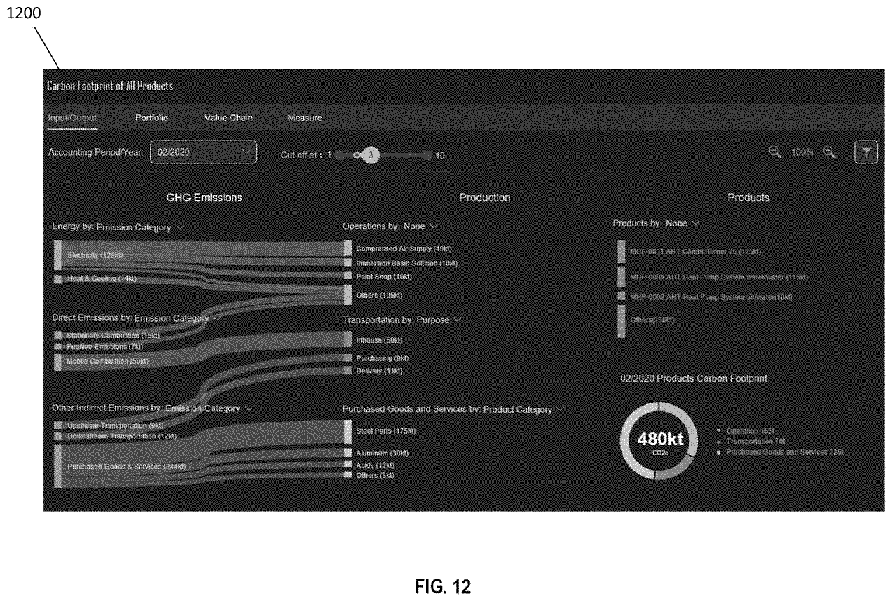

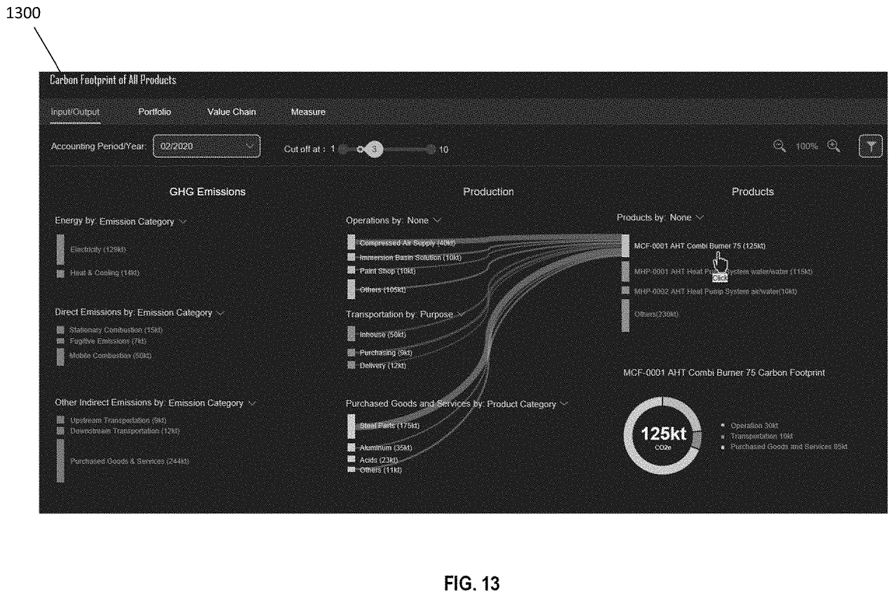

FIG. 12 depicts an example of a user interface 1200 which may present results of the GHG allocations disclosed herein. The user interface 1200 depicts an example of how the GHG emissions are allocated to different operations during production, while FIG. 13 depicts how the emissions are allocated emissions and a type of Energy Carrier (e.g., “GAS_Natural Gas”). For example, FIG. 12 depicts how the GHG CO2 emissions are allocated to the various steps in the production process, such as the oven, melter, assembler, warehousing, etc. In the example of FIG. 12, a Sankey diagram is used, although other types of diagrams are used as well. At the user interface 1200, the first column shows the actual emissions in CO2 equivalents that result from any product related activity in the given period of time and structured by the GHG Protocol emission categories in total and on corporate level. The total tons of CO2 emissions of all line items in this column is based on quantity produced in that period, rather than quantity sold. It comprises scope 1 and 2 emissions as required by the GHG Corporate Standard, plus product-related scope 3 emissions as required by GHG Scope 3 standard. FIG. 13 on the other hand provides a user interface 1300 showing how the GHG CO2 emissions are allocated to a specific product.

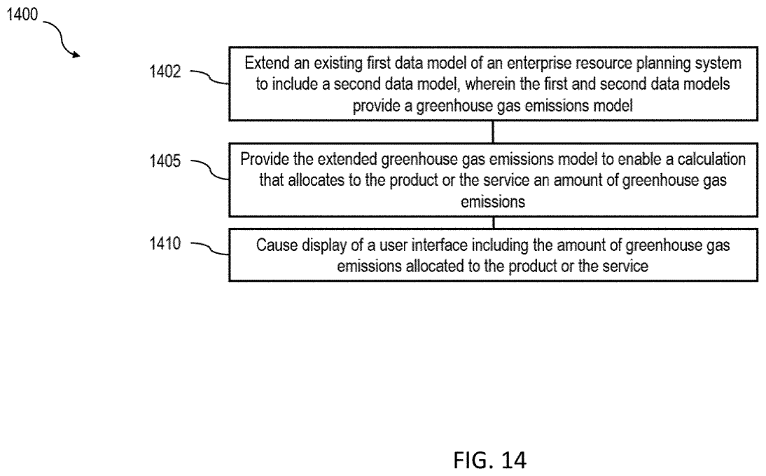

FIG. 14 shows a process flow for allocating GHG emissions based on a GHG data flow model.

At 1402, a first data model of an ERP system is extended to include a second data model, such that the first and second data models provide a greenhouse gas emission model. The ERP system may include existing models that model the transactions associated with a product or service. For example, an existing first data model may include transactional data associated with a product or a service. The transaction data may include goods receipt from supplier per material, internal service confirmations from production, and the like. The second data model may include energy data and flows associated with the product or the service. Referring again to FIG. 1 for example, the ERP system's data flows and nodes 102A-G (as indicated by the solid lines) are extended to include the energy nodes and data flows 104A-B (as indicated by the dotted lines). As noted, the ERP system's data flows and nodes 102A-G may also be extended to include and thus model emissions that occur during the life cycle of a material or a product after the sale, such as emissions related to distribution, storage, product use, disposal, and/or end-of-life. As a result, holistic GHG data model (e.g., the extended ERP data model) may provide a single, unified model of the physical value chain and the energy flows that allows allocating resources including the GHG emissions at a product level using the existing ERP system infrastructure from cradle to grave, for example.

At 1405, the greenhouse gas emission model may be provided. After being extended to include the second model, the greenhouse gas emission model including the first and second data model is ready to be run and thus may be used to calculate C02 emissions, which are allocated to a product or a service level.

At 1410, the allocated emissions may be presented on a user interface. For example, the allocated C02 emissions calculated based on the greenhouse gas emission model (e.g., the ERP data models extended to include data models associated with GHG emissions) may be presented in predetermined format, such as a Sankey diagram as depicted at FIGS. 12 and 13.

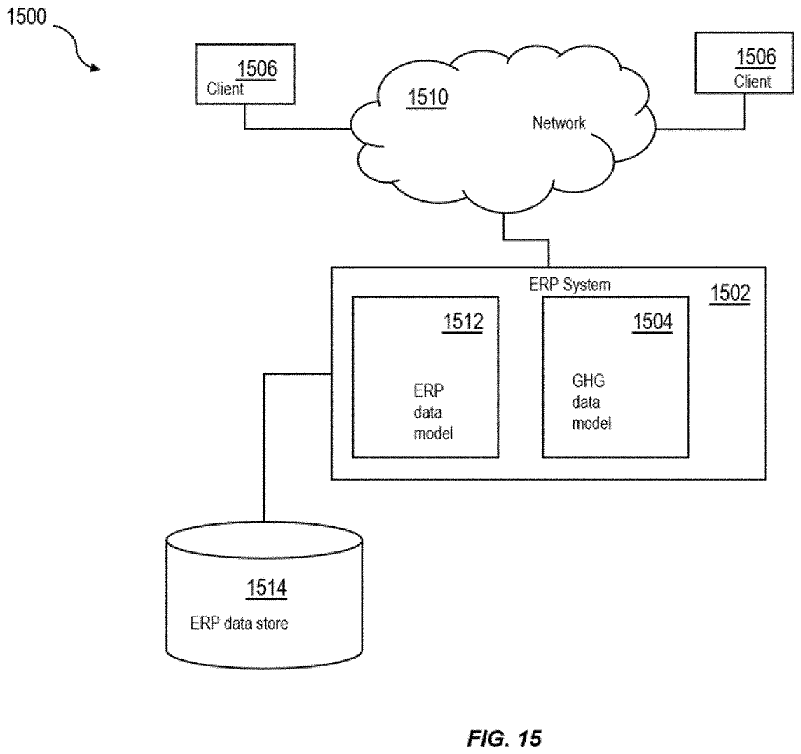

FIG. 15 illustrates an exemplary system 1500 in which a computing system such as an ERP system 1502 executes one or more modules, software components, or the like including an ERP data model 1512 and an GHG data model 1504, according to some implementations of the current subject matter. The ERP system 1502 may be accessible to local users of the computing system 1502 as well as to remote users accessing the computing system 1502 from one or more client device machines 1506 over a network connection 1510. One or more user interface screens produced by the one or more modules 1512 and 1504 may be caused to be displayed to a user, either via a local display or via a display associated with one of the client machines 1506. Data units of the ERP system 1502 may be stored in a persistence layer 1514.

Aspects of the subject matter described herein can be embodied in systems, apparatus, methods, and/or articles depending on the desired configuration. In particular, various implementations of the subject matter described herein can be realized in digital electronic circuitry, integrated circuitry, specially designed application specific integrated circuits (ASICs), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs, which can also be referred to programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, the subject matter described herein can be implemented on a computer having a display device, such as for example a cathode ray tube (CRT) or a liquid crystal display (LCD) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

The subject matter described herein can be implemented in a computing system that includes a back-end component, such as for example one or more data servers, or that includes a middleware component, such as for example one or more application servers, or that includes a front-end component, such as for example one or more client computers having a graphical user interface or a Web browser through which a user can interact with an implementation of the subject matter described herein, or any combination of such back-end, middleware, or front-end components. A client and server are generally, but not exclusively, remote from each other and typically interact through a communication network, although the components of the system can be interconnected by any form or medium of digital data communication. Examples of communication networks include, but are not limited to, a local area network (“LAN”), a wide area network (“WAN”), and the Internet. The relationship of client and server arises by virtue of computer programs running on the respective computers and having a client-server relationship to each other.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described above can be directed to various combinations and sub-combinations of the disclosed features and/or combinations and sub-combinations of one or more features further to those disclosed herein. In addition, the logic flows depicted in the accompanying figures and/or described herein do not necessarily require the particular order shown, or sequential order, to achieve desirable results. The scope of the following claims may include other implementations or embodiments.

What is claimed:

1. A method comprising:
extending an existing first data model of an enterprise resource planning system to include a second data model, wherein the first and second data models provide a greenhouse gas emissions model;
providing the extended greenhouse gas emissions model to enable a calculation that allocates to the product or the service an amount of greenhouse gas emissions; and
causing display of a user interface including the amount of greenhouse gas emissions allocated to the product or the service.

2. The method of claim 1, wherein the first data model includes transactional data associated with the product or the service.

3. The method of claim 2, wherein the second data model includes energy data and flows associated with the product or the service.

4. The method of claim 3, wherein the second data model includes emissions that occur during a least a portion of a lifecycle of the product or the service, the portion including emissions related to distribution, storage, usage, and/or disposal.

5. The method of claim 4, wherein the first and second data models are configured to form a directed graph to enable the calculation that allocates to the product or the service the amount of greenhouse gas emissions.

6. The method of claim 1, wherein the user interface is configured as a Sankey diagram.

7. A system comprising:
at least one processor; and
at least one memory including program code which when executed causes the system to provide operations comprising:
extending an existing first data model of an enterprise resource planning system to include a second data model, wherein the first and second data models provide a greenhouse gas emissions model;
providing the extended greenhouse gas emissions model to enable a calculation that allocates to the product or the service an amount of greenhouse gas emissions; and
causing display of a user interface including the amount of greenhouse gas emissions allocated to the product or the service.

8. The system of claim 7, wherein the first data model includes transactional data associated with the product or the service.

9. The system of claim 8, wherein the second data model includes energy data and flows associated with the product or the service.

10. The system of claim 9, wherein the second data model includes emissions that occur during a least a portion of a lifecycle of the product or the service, the portion including emissions related to distribution, storage, usage, and/or disposal.

11. The system of claim 10, wherein the first and second data models are configured to form a directed graph to enable the calculation that allocates to the product or the service the amount of greenhouse gas emissions.

12. The system of claim 7, wherein the user interface is configured as a Sankey diagram.

13. The system of claim 7, wherein the system is comprised in or comprised an enterprise resource planning system.

14. A non-transitory computer-readable storage medium including program code which when executed by at least one processor causes operations comprising:
extending an existing first data model of an enterprise resource planning system to include a second data model, wherein the first and second data models provide a greenhouse gas emissions model;
providing the extended greenhouse gas emissions model to enable a calculation that allocates to the product or the service an amount of greenhouse gas emissions; and
causing display of a user interface including the amount of greenhouse gas emissions allocated to the product or the service.

15. The non-transitory computer-readable storage medium of claim 14, wherein the first data model includes transactional data associated with the product or the service.

16. The non-transitory computer-readable storage medium of claim 15, wherein the second data model includes energy data and flows associated with the product or the service.

17. The non-transitory computer-readable storage medium of claim 16, wherein the second data model includes emissions that occur during a least a portion of a lifecycle of the product or the service, the portion including emissions related to distribution, storage, usage, and/or disposal.

18. The non-transitory computer-readable storage medium of claim 17, wherein the first and second data models are configured to form a directed graph to enable the calculation that allocates to the product or the service the amount of greenhouse gas emissions.

19. The non-transitory computer-readable storage medium of claim 14, wherein the user interface is configured as a Sankey diagram.
